(19) United States
(12) Patent Application Publication
(10) Pub. No.: US 2006/0159657 A1
(43) Pub. Date: Jul. 20, 2006

(54) Formulations of lymphokines and method of use thereof for local or both local and systemic control of proliferative cell disorders

(75) Inventors: Kirk D. Fowers, Centerville, UT (US); Gaylen M. Zentner, Salt Lake City, UT (US); Miroslav Baudys, Lilburn, GA (US); Maria Jurek, Sandy, UT (US); Wolfram Samlowski, Salt Lake City, UT (US)

Correspondence Address: THORPE NORTH & WESTERN, LLP., 8180 SOUTH 700 EAST, SUITE 200, SANDY, UT, 84070, US

(73) Assignee: MacroMed, Incorporated

(21) Appl. No.: 11/386,156

(22) Filed: Mar. 21, 2006

Related U.S. Application Data
(63) Continuation-in-part of application No. 10/172,805, filed on Jun. 13, 2002, now abandoned.
(60) Provisional application No. 60/298,594, filed on Jun. 14, 2001.

(51) Int. Cl. A61K 38/20; A61K 9/14

(52) U.S. Cl. 424/85.2; 424/486

(57) ABSTRACT

Therapeutic formulations comprising an effective amount of IL-2 or other lymphokine and a biodegradable polymeric carrier having reverse gelation properties and the methods of use thereof for local or both local and systemic control of proliferative cell disorders is disclosed. The formulation can be administered intratumorally/peritumorally and forms an IL-2 containing depot. The IL-2-containing depot provides for continuous, prolonged release of IL-2 sufficient to stimulate the production of cytotoxic T lymphocytes which function both locally and systemically, without causing unacceptable side effects.

### PRIORITY CLAIM

This is a continuation-in-part of U.S. patent application Ser. No. 10/172,805 filed on Jun. 13, 2002, which claims the benefit of U.S. Provisional Application No. 60/298,594, filed on Jun. 14, 2001.

### BACKGROUND

The present invention is in the field of localized sustained delivery of a lymphokine into a warm blooded animal. More particularly, this invention relates to therapeutic formulations and the methods of use thereof for local or both local and systemic control of proliferative cell disorders.

Recent advances in understanding the biology of the immune system have lead to the identification of important modulators of immune responses, generally called cytokines or if produced by lymphocytes, lymphokines. Rosenberg, Steven A. “The Immunotherapy and Gene Therapy of Cancer.” J. Clin. Oncology 10:180-199 (1992). Cytokines are small proteins secreted primarily, but not exclusively, by cells of the immune system that promote the proliferation and/or differentiative functions of other cells. Examples of cytokines include interleukins, interferons, hematopoietic colony stimulating factors (CSF), and proinflammatory factors such as tumor necrosis factor (TNF). The administration of cytokines and related immunomodulators has resulted in objective tumor responses in patients with various types of neoplasms. Sama, Gregory et al., “A Pilot Study of Intralymphatic Interleukin-2. II. Clinical and Biological Effects”, J. Biol. Response Modifiers, 9:81-86 (1990). However, current modes of cytokine administration are frequently associated with toxicities that limit the therapeutic value of these agents, which is due in part to a general activation of the immune system.

Interleukin-2 (IL-2), a lymphokine produced by normal peripheral blood lymphocytes that induces proliferation of antigen or mitogen stimulated T cells after exposure to plant lectins, antigens, or other stimuli, was first described by Morgan, D. A., et al., Science (1976), 193:1007-1008. IL-2 acts on the three major types of lymphocytes: T cells, B cells, and NK cells, stimulating them to proliferate and augmenting their differentiative functions. IL-2 potentiates both innate or natural host defenses by stimulating NK cells and antigen-specific acquired immune reactivity by stimulating T cells and B cells. IL-2 was initially made by cultivating human peripheral blood lymphocytes (PBL) or other IL-2-producing cell lines, described in, for example, U.S. Pat. No. 4,401,756. Recombinant DNA technology has provided an alternative to PBLs and other cell lines for producing IL-2. Taniguchi, T. et al., Nature (1983), 302:305-310 and Devos, R., Nucleic Acids Research (1983), 11:4307-4323 have reported cloning the human IL-2 gene and expressing it in microorganisms.

U.S. Pat. No. 4,518,584 describes and claims mutated proteins of IL-2 in which the cysteine normally occurring at position 125 of the wild-type or native molecule has been replaced with a neutral amino acid, such as serine or alanine. U.S. Pat. Nos. 4,530,787 and 4,569,790 disclose and claim methods for purifying recombinant native IL-2 and muteins thereof, as well as the purified form of IL-2. U.S. Pat. No. 4,604,377 discloses an IL-2 composition suitable for reconstitution in a pharmaceutically acceptable aqueous vehicle composed of oxidized microbially produced recombinant IL-2.

In response to tumor antigens, a subset of lymphocytes, termed helper T-cells, secrete small quantities of IL-2. This IL-2 acts locally at the site of tumor antigen stimulation to activate cytotoxic T-cells and natural killer cells that mediate systemic tumor cell destruction. Intravenous, intralymphatic and intralesional administration of IL-2 has resulted in clinically significant responses in some cancer patients. It has been shown that systemic administration of recombinant IL-2 in high doses causes regression of established metastatic cancers in mice, and stimulates lymphokine-activated killer cells, and tumor-infiltrating lymphocytes in humans. Rosenberg et al., J. Exp. Med. (1985) 161:1169-1188; Rosenberg, S. et al., New Eng. J. Med. (1985)313:1485-1492; Rosenberg et al., Science (1986) 233:1318-1321). However, severe toxicities (hypotension and edema) limit the dose and efficacy of intravenous and intralymphatic IL-2 administration. Hoover, H. C. Jr. et al. Cancer Res. (1984), 44(4):1671-6. The toxicity of systemically administered lymphokines is not surprising as these agents mediate local cellular interactions and they are normally secreted in only very small quantities. Additionally, other lymphokines, such as interleukin-4 (IL-4), alpha interferon α-INF) and gamma interferon (γ-INF) have been used to stimulate immune responses to tumor cells. As with IL-2, the current modes of administration have adverse side effects.

To circumvent the toxicity of systemic IL-2 administration several investigators have examined intralesional injection of IL-2. This approach eliminates the toxicity associated with systemic IL-2 administration. However, multiple intralesional injections are required to optimize therapeutic efficacy due to the short half-life when IL-2 is administered without a means to control the release. Bubenik, J. et al., Immunology Letters. 23:287-292 (1989/1990); Borden, Ernest C., and Sondel, Paul M Cancer. 1990 Feb. 1; 65(3 Suppl):800-14; Rosenberg, Steven A. et al., 108:853-864 (1988). Hence, these injections are impractical for many patients, particularly when tumor sites are not accessible for injection without potential morbidity. One example of a tumor that has significant morbidity following administration of free IL-2, and which is not readily accessible for repeat injections, is the brain

U.S. Pat. No. 6,045,788 discloses that low doses of natural and recombinant IL-2, may be continuously administered to patients for prolonged periods of time, e.g. longer than three months, in order to activate and/or stimulate their immune system without causing substantial toxicity. However, frequent administration for prolonged periods of time is required, or, in the alternative, an optimum sustained release formulation that is not described in the patent is needed.

A variety of administration techniques have been evaluated that decrease the total dose administered to reduce the number and severity of side effects. Examples include: subcutaneous Atzpodien, J. et al., Mol Biother. 2:18-26 (1990) and pulmonary Huland, E. et al., Hepato-Gastroenterology 46 Supplement:1257-1262 (1999) routes. While these techniques have reduced the toxicity of the treatment, the therapy continues to require frequent admission due to the short half-life and the number of patients experiencing a durable objective response is decreased compared to high-dose therapy. The half-life of IL-2 is prolonged with both methods, but is still limited, in comparison to the present invention, which results in a diminished response rate due to the decreased concentration at the site of action. Eton, O. Cancer. 95:127-34 (2002) and Kirchner, GI. Br J Clin Pharmacol. 46:5-10 (1998). While frequent administration with a lower concentration of IL-2 mitigates the toxicity associated with higher dose administrations, the activation of T-cells and NK cells is diminished and sustained release of IL-2 to promote cytotoxicity against target cells is intermittent rather than continuous. The present invention more closely mimics the natural activation at the site of action and recruitment of additional immunomodulator cells by the release of local IL-2.

In addition to the potential toxicity, another problem of systemic administration of IL-2 is rapid renal elimination. When IL-2 is given intravenously it is eliminated rapidly with an initial elimination half-life and terminal elimination half-life of 6 to 12 minutes and 40 to 80 minutes, respectively. Sands H, Loveless SE. Biodistribution and pharmacokinetics of recombinant, human ¹²⁵I-interleukin-2 in mice. Int J immunopharmacol 1989; 11:411-6 and Lotze M T, Matory Y L, Ettinghausen S E, et al. In vivo administration of purified human interleukin-2. II. Half-life, immunologic effects, and expansion of peripheral lymphoid cells in vivo with recombinant IL-2 J Immunol 1985:135:2865-75.

Therefore, there is a need for a formulation of IL-2 that forms an IL-2 containing depot after being administered locally to a warm-blooded animal. The IL-2 containing depot provides a locally high concentration and continuous, prolonged release of IL-2 sufficient to locally stimulate the production of cytotoxic T lymphocytes (CTTL's) which function locally or both locally and systemically, without causing unacceptable side effects. The prolonged release should include a suitable pharmacokinetic profile resulting in activation of tumor infiltrating lymphocytes, sustained release sufficient to attract additional CTTL's to the tumor site, and an enhancement in the number of durable complete responses to therapy. An optimum material for use as an injectable or implantable polymeric drug carrier in the formulation of the present invention should be biodegradable, compatible with the drugs to be used, and allow fabrication with simple, safe solvents, such as water, and not require additional polymerization or other covalent bond forming reactions following administration.

### SUMMARY OF THE INVENTION

The invention provides a therapeutic formulation comprising a biodegradable block copolymeric drug carrier having thermally reversible gelation properties and an effective amount of a lymphokine, said formulation can be administered intratumorally/peritumorally and forms a lymphokine containing depot. The lymphokine-containing depot provides for a locally high concentration and continuous, prolonged release of lymphokines such as IL-2 sufficient to simulate the production of cytotoxic T lymphocytes which function locally or both locally and systemically, without causing any significant undesirable systemic side effects. The formulation is broadly applicable to administration at sites typically inaccessible to systemic administration, i.e., the brain. The pharmacokinetic profile results in a plasma half-life of greater than 24 hours, which provides a prolonged concentration gradient for attraction of additional CTTL's to the tumor site. The formulation results in a prolonged plasma half-life that serves to attract additional immunological active cells, while simultaneously reducing the amount of IL-2 entering systemic circulation and subsequently being excreted. The term “lymphokine” refers to any agent having IL-2 or other interleukin activity, including natural, recombinant and mutated IL-2 or other interleukins, analogs and derivatives thereof. Preferred lymphokines can be selected from the group consisting of interleukin-2 (IL-2), interleukin-4, interleukin-12 and their derivatives.

The present invention also provides a method of converting a highly toxic drug, i.e. IL-2 or other lymphokines, into a fully effective drug with minimal unacceptable side effects by incorporating IL-2 or other lymphokines into a biodegradable drug carrier capable of forming a drug containing depot after local administration. Preferably, the biodegradable drug carrier has reverse thermal gelation properties, namely, such drug carriers are in a liquid state below body temperature but transition to a gel or solid state as they are warmed, and exist as gels at or about body temperature. In the remainder of this disclosure, the narrative will generically refer to IL-2, but the present teachings, unless otherwise indicated, extend to the remainder of the agents encompassed herein. Preferably, the IL-2 is a recombinant human IL-2.

The invention further provides a method for therapeutic or prophylactic treatment of the tumor burden in a warm blooded animal host, comprising locally administering to said host a formulation comprising an effective amount of IL-2, or other lymphokines, formulated with a biodegradable drug carrier having reverse thermal gelation properties.

The present invention can be used to treat/cure proliferative cellular disorders such as cancer, or warts. The formulations of the present invention, when administered locally, provide for sustained local release of high levels of IL-2, or other lymphokines (still a low total dose based on systemic exposure), directly to the diseased tissue to be treated, i.e. tumors or warts, stimulating the production of cytotoxic T-lymphocytes that attack the diseased tissue at both the local site and, following local activation, throughout the body as well, without causing unacceptable systemic side effects. Without the presence of the drug carriers used in the present formulation, the IL-2 is largely ineffective due to rapid clearance from the injection site before CTTL stimulation occurs. Because the drug, namely IL-2 or other lymphokines, stimulates the body's own defenses to attack the diseased tissues, the potential problem of drug resistance is eliminated. Therefore, the present invention provides formulations and methods of use for the treatment of many types of local and metastatic cancer from simple, low-dose local injections. The formulations of the present invention may be administered at intervals ranging from daily to monthly. The formulations of the present invention also provide for local, or both local and systemic anti-cancer therapy from the localized injection of a dose of IL-2 that would be sub-therapeutic if administered systemically.

The present invention also provides a therapeutic formulation which can be administered intratumorally/peritumorally and forms a drug containing depot with a desirable pharmacokinetic(PK) profile. For example, the initial release of IL-2 from the depot is between 0.1 and 50% of the administered dose, preferred is 5-40%, and most preferred is 10-30%. IL-2 release from the system is within the range from 1-90 days, preferred is 3-30 days, and most preferred is 5-14 days. The plasma half-life is from 6-72 hours, preferred is 12-48 hours, and most preferred is from 18-24 hours. Additionally, the excretion of IL-2 is reduced. Currently urine excretion for subcutaneous administration is approximately 40%, which is reduced to below 30% in the present invention. Similarly, excretion via the fecal route is approximately 2%, which is reduced to less than 0.5% in the present invention.

The present invention also provide an IL-2 formulation which can be administered at locations within the body that would be difficult to administer IL-2 daily, i.e., the brain, where a single intracranial administration would allow IL-2 to be released for an extended period of time. It would be impractical to administer IL-2 using conventional techniques within the brain. Daily or multiple daily injections are impractical, and continuous infusion would cause generalized inflammation. ReGel®/IL-2 controls the release and allows the area of action to be focused at the injection site. It also creates a concentration gradient that attracts cells to the brain and to a specific location. Another facet of the injections into the brain is that side effects unique to the brain would be minimized. Inflammation is a common side effect of IL-2 administration, which was reduced when ReGel/IL-2 was administered.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1A illustrates IL-2 release in vitro from the IL-2 formulation, according to Example 1, as analyzed and monitored by ELISA.

FIG. 1B illustrates IL-2 release in vitro from the IL-2 formulation, according to Example 1, as analyzed and monitored by a cell proliferation assay.

FIG. 2 illustrates the cytotoxicity assay results of the IL-2 formulation according to Example 1, measured by percentage of specific cell lysis.

FIG. 3 illustrates the effects of administration of the IL-2 formulation, according to Example 1, on fibrosarcoma tumor growth as measured by change in tumor size over time.

FIG. 4 illustrates IL-2 release in vitro from the IL-2 formulation, according to Example 4, monitored by ELISA.

FIG. 5 illustrates the dose escalation effect of administration of the IL-2 formulation, according to Example 4, on fibrosarcoma tumor growth as measured by change in tumor size over time.

FIG. 6 illustrates the effect of repetitive administration of the IL-2 formulation, according to Example 4, on RENCA tumor growth as measured by change in tumor size over time.

FIG. 7 illustrates the effect of repetitive administration of the IL-2 formulation, according to Example 4, on mouse survival rate in a MetA fibrosarcoma tumor model.

FIG. 8 illustrates the effect of repetitive dosing of the IL-2 formulation, according to Example 4, on mouse survival rate in a B16 melanoma tumor model.

FIG. 9 illustrates activation of systemic immunity by IL-2 released from the IL-2 formulation, according to Example 9, as analyzed and monitored by inhibition of tumor growth in comparison to subcutaneous injection of recombinant IL-2.

FIG. 10A illustrates retention of IL-2 from the formulation at the peitumoral injection sites, according to Example 10, as analyzed and monitored by radioactivity count.

FIG. 10B illustrates the blood concentration of IL-2 from the IL-2 formulation, according to Example 10, as analyzed and monitored by radioactivity count.

FIG. 10C illustrates the concentration of IL-2 recovered from the kidneys from the IL-2 formulation, according to Example 10, as analyzed and monitored by radioactivity count.

FIG. 10D illustrates the concentration of IL-2 eliminated through the urine from the IL-2 formulation, according to Example 10, as analyzed and monitored by radioactivity count.

FIG. 10E illustrates the concentration of IL-2 eliminated through the feces from the IL-2 formulation, according to Example 10, as analyzed and monitored by radioactivity count.

FIG. 11 illustrates the effect of a single administration of the IL-2 formulation, according to Example 12, on mouse survival rate in an intracranial B-16 melanoma tumor model.

Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENT(S)

Before the present therapeutic formulations and methods of use thereof for drug delivery, and in particular for IL-2 delivery, are disclosed and described, it is to be understood that this invention is not limited to the particular configurations, process steps, and materials disclosed herein. Such configurations, process steps, and materials may vary somewhat, which are intended to be within the scope of the invention. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a”, “an”, and “the”, includes plural referents unless the context clearly dictates otherwise. Thus, for example, reference to a composition for delivering “a drug” includes reference to one, two, or more drugs. In describing and claiming the present invention, the following terminology will be used in accordance with the definitions set forth below.

“Parenteral” shall mean intratumoral, peritumoral, intralesional, perilesional, intrathecal, intraperitoneal, intracranial and intra-abdominal.

The term “cell proliferative disorders” includes disorders of cell proliferation and differentiation commonly considered neoplastic or malignant, such as cancers. Cancer includes, for example, carcinomas, melanomas, myelomas, sarcomas, and the like. The term cancer also includes pre-cancerous tissues and cells that are known to progress into true cancer if left untreated. Other examples of cell proliferative disorder are warts.

The term “therapeutic” treatment refers to administration to patients of a drug, particularly IL-2, after the patient has developed cancer, (i.e., after a tumor burden has been determined), as measured by any means in the art, with a resultant decrease or elimination of the existing tumor burden being the goal.

The term “prophylactic” treatment refers to such administration to prevent recurrence of the cancer after a therapeutic treatment has been administered.

The term “pharmacologically effective amount” refers to the amount of each active component of the method or composition herein that is sufficient in showing a meaningful patient benefit, i.e., prolongation of life and/or reduction of disease and/or improvement in any clinically significant way. When the effective amounts defined herein are employed, more efficacy is obtained using the combination than using either component alone. As applied to an individual active ingredient administered alone, the term refers to that ingredient alone; when combinations are used, the term refers to combined amounts in the preparation that result in the therapeutic or prophylactic effect.

The term “recombinant” refers to drugs produced by recombinant DNA techniques wherein in the particular case of IL-2 the gene coding for the IL-2 is cloned by known recombinant DNA technology.

The term “pharmaceutically acceptable” refers to a carrier medium that does not interfere with the effectiveness of the biological activity of the drug and that is not toxic to the host to which it is administered.

“Gelation temperature” means the temperature at which the biodegradable block copolymer undergoes reverse thermal gelation. In other words, the temperature below which the block copolymer is apparently soluble or exists as a uniform colloidal system in water and exists as a free-flowing fluid, and above which the block copolymer undergoes phase transition to increase in viscosity or to form a semi-solid gel. The terms “gelation temperature” and “reverse thermal gelation temperature” or the like shall be used interchangeably in referring to the gelation temperature.

“Polymer solution”, “aqueous solution”, “solution”, “homogeneous solution” and the like, when used in reference to a biodegradable block copolymer contained in such solution, shall mean a water based solution having such block copolymer dissolved or in a uniform colloidal state therein at a functional concentration, and maintained at a temperature below the gelation temperature of the block copolymer. They shall include water without additives or aqueous solutions containing additives or excipients such as pH buffers, components for tonicity adjustment, antioxidants, preservatives, drug stabilizers, etc., as commonly used in the preparation of pharmaceutical formulations.

“Reverse thermal gelation” is the phenomenon whereby a solution of a block copolymer spontaneously increases in viscosity, and in many instances transforms into a semisolid gel, as the temperature of the solution is increased above the gelation temperature of the copolymer. For the purposes of the invention, the term “gel” includes both the semisolid gel and the high viscosity state that exists above the gelation temperature. When cooled to below the gelation temperature, the gel spontaneously reverses over a period of a few minutes to several hours to reform the lower viscosity free-flowing fluid. All interactions to create the gel are physical in nature and do not involve the formation or breaking of covalent bonds.

“Drug delivery liquid” or “drug delivery liquid having reverse thermal gelation properties” shall mean polymer solutions that contain a drug (the drug per se can either be dissolved, dispersed or colloidal) suitable for administration to a warm-blooded animal. The drug containing polymer solution forms a gelled drug depot when the temperature is raised to or above the gelation temperature of the drug delivery liquid.

“Depot” means a localized site in the body containing concentrated active agents or drugs. Examples of formulations that form depots are gels, implants, microspheres, matrices, particles, etc.

“Gel” means the semi-solid phase that spontaneously occurs as the temperature of the “polymer solution” or “drug delivery liquid” is raised to or above the gelation temperature of the block copolymer.

“Gel mixture” or “mixture of triblock copolymers” refers to a reverse thermal gelation system comprising two or more ABA or BAB triblock copolymer components. The mixture can be made either by simply mixing two or more individually synthesized triblock copolymer components, or by synthesizing two or more types of copolymer systems in one synthesizing vessel. The mixture prepared by the above two processes may be combined with water to form a polymer solution that may have the same or different gelation properties and gel qualities.

“Solution”, “solubilized”, “dissolved” and all other terms that refer to a solution or dissolved state includes a homogeneous solution, micellar solution, or any apparently uniform colloidal state such as an emulsion or a suspension.

“Biodegradable” means that the block copolymer can chemically break down or degrade within the body to form nontoxic components. The rate of degradation can be the same or different from the rate of drug release.

“Drug” shall mean any organic or inorganic compound or substance having biological or pharmacological activity that can be adapted or used for a therapeutic purpose.

“Peptide,” “polypeptide,” “oligopeptide” and “protein” shall be used interchangeably when referring to peptide or protein drugs and shall not be limited as to any particular molecular weight, peptide sequence or length, field of bioactivity or therapeutic use unless specifically stated.

“Interleukin” shall mean any protein/polypeptide agent or its derivative, analog or mimetic, having interleukin activity, particularly IL-2, IL-4 and IL-12 activities, including natural and recombinant IL-2, IL-4 and IL-12, pharmaceutically-acceptable fusion proteins of natural and recombinant interleukins, derivatives and mixtures thereof.

“hlL-2” refers to a protein exhibiting the spectrum of activities characterizing human interleukin-2. Specifically, the protein must be capable of stimulating the proliferation of hlL-2 dependent cytolytic and helper T cell lines, as set forth in the standard assays of S. Gillis et al, J. Immunol. (1978) 120:2027-2032 and of J. Watson, J. Exp. Med. (1979) 150:1510-1519. Modified IL-2, IL-4 and IL-12 are also included in this definition so long as biological activity is not destroyed thereby.

“PLGA” shall mean a copolymer or copolymer radicals derived from the condensation copolymerization of lactic acid and glycolic acid, or, by the ring opening copolymerization of lactide and glycolide. The term lactic acid and lactate are used interchangeably; glycolic acid and glycolate are also used interchangeably.

“PLA” shall mean a polymer derived from the condensation of lactic acid or by the ring opening polymerization of lactide.

“PGA” shall mean a polymer derived from the condensation of glycolic acid or by the ring opening polymerization of glycolide.

“Biodegradable polyester or poly(ortho ester)s” refers to any biodegradable polyester or poly(ortho ester), wherein the polyesters are preferably synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydroxybutyric acid, malic acid, and copolymers thereof.

“ReGel®” is a tradename of MacroMed Incorporated for a class of low molecular weight, biodegradable block copolymers having reverse thermal gelation properties as described in U.S. Pat. Nos. 6,004,573, 6,117949, 6,201,072, and 6,287,588, hereby incorporated by reference. It also includes biodegradable polymeric drug carriers disclosed in pending U.S. patent application Ser. Nos. 09/906,041, 09/559,799 and 10/919,603 hereby incorporated by reference. The biodegradable drug carrier comprises ABA-type or BAB-type triblock copolymers or mixtures thereof, wherein the A-blocks are relatively hydrophobic and comprise biodegradable polyesters or poly(ortho ester)s, and the B-blocks are relatively hydrophilic and comprise polyethylene glycol (PEG), said copolymers having a hydrophobic content of between 50.1 to 83% by weight and a hydrophilic content of between 17 to 49.9% by weight, and an overall block copolymer molecular weight of between 2000 and 8000 Daltons. The drug carriers exhibit water solubility at temperatures below normal mammalian body temperatures and undergo reversible thermal gelation to then exist as a gel at temperatures equal to physiological mammalian body temperatures. The biodegradable, hydrophobic A polymer block comprises a polyester or poly (ortho ester), wherein the polyester is synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxyhexanoic acid, γ-butyrolactone, γ-hydroxybutyric acid, δ-valerolactone, δ-hydroxyvaleric acid, hydroxybutyric acids, malic acid, and copolymers thereof and having an average molecular weight of between about 600 and 3000 Daltons. The hydrophilic B-block segment is preferably polyethylene glycol (PEG) having an average molecular weight of between about 500 and 2200 Daltons.

The concentration at which the block copolymers are water soluble at temperatures below the gelation temperature may be considered as the functional concentration. Generally speaking, block copolymer concentrations of as low as 3% and up to about 50% by weight can be used and still be functional. However, concentrations in the range of about 5 to 40% by weight are preferred and concentrations in the range of about 10 to 30% by weight are most preferred. At the lower functional concentration ranges, phase transition may result in the formation of a weak gel or viscous liquid, however these systems are still functional for purposes of the invention.

The formulation of the present invention may also comprise a reconstitution enhancing and enabling agent comprising a liquid polyethylene glycol (PEG), or a mixture of PEG and a PEG derivative, said PEG or PEG derivative having a molecular weight of 150 to 1100 Daltons. The PEG derivative is comprised of PEG and a member selected from the group consisting of D,L-lactide, D-lactide, L-lactide, D,L-lactic acid, D-lactic acid, L-lactic acid, glycolide, glycolic acid and copolymers thereof. The PEG derivative can also be a member represented by R¹—CO—O-(PEG)-CO—R² or R¹—O-(PEG)-R² wherein R¹ and R² are independently members selected from the group consisting of H and C₁ to C₁₀ alkyl groups.

The mixture of the biodegradable copolymer and the drug, such as IL-2, may be prepared as an aqueous solution or uniform colloid of the copolymer below the gelation temperature to form an IL-2 delivery liquid wherein IL-2 may be either partially or completely dissolved. This IL-2 delivery liquid is then administered via local routes of delivery, such as intratumoral or peritumoral, to a patient whereupon it undergoes reversible thermal gelation since body temperature will be above the gelation temperature. This substantially reduces or in many cases eliminates the systemic toxicity of IL-2 and maximizes the immune system response to cancer cells. Other potential applications of the IL-2 formulations of the present invention include administration locally at the site of surgical intervention/tumor excision to kill cancer cells remaining after surgery. Other possible local routes of delivery to treat malignant tumors include intraperitoneal administration for tumors/metastases localized in the peritoneum, and intracranial administration for tumors localized in the skull. The latter localization is representative of sites where routes other than parenteral would not deliver sufficient concentrations for activity without increasing systemic side effects. Alternate delivery mechanisms include intravenous, introperitoneal, transdermal, buccal, inhalation, and similar routes of administration.

The present invention also provides a general method for converting highly toxic cytokines and related drugs, such as lymphokines, including IL-2, IL-4, IL-12 and their derivatives and mimetics, into effective anticancer agents, while minimizing negative side effects. The formulations of the present invention provide for sustained delivery of the drug from a local depot formed intratumorally/peritumorally at a single or several localized tumor site(s), and which induce local or both local and systemic immune responses.

By forming a local sustained delivery drug depot intratumorally/peritumorally, a relatively high local cytokine steady state concentration at the tumor site is achieved. The high local concentration of IL-2 stimulates the activation and production of tumor specific CTTL's that attack tumor tissue both locally as well as throughout the body, while the overall systemic IL-2 level is low enough to avoid any significant unacceptable side effects. While the systemic levels are low enough to avoid side effects, the release of IL-2 from the depot provides a concentration gradient that cells sensitive to IL-2, i.e., T-cells and NK cells, can track to the site of release for days to months. Therefore, it provides an enhanced immunologic response due to the continuous release of IL-2, in contrast to immediate release delivery systems which result in IL-2 half-lives of minutes to hours.

The formulation and method of the present invention are useful for treatment of any tumors that present as a manifestation of a cancer disease that can be formed by any malignant or pre-cancerous tissue, including carcinomas, sarcomas, melanomas, myelomas and the like. The formulations and methods of the present invention provide a way of converting a highly toxic drug, particularly a cytokine such as IL-2, IL-4, IL-12 and their derivatives and mimetics, into effective therapeutic formulations without significant undesirable side effects. Even though the overall systemic lymphokine dose administered is lowered by an order of magnitude as compared to the dose of IL-2 or other lymphokine required for systemic treatment, the formulations of the present invention still provide for better or comparable pharmacological effects. This is based on the altered release profile, which can be comprised of a well-defined burst and a prolonged release profile.

In one embodiment of the present invention, IL-2 is incorporated in a biodegradable block copolymeric drug carrier having reverse thermal gelation properties (ReGel) in a soluble form or as a suspension or other colloidal form and kept below its gelation temperature. After being administered intratumorally or peritumorally to a patient or a diseased animal (warm blooded mammalian host) whose bodies' temperature is above the ReGel gelation temperature, the formulation forms local depots which can provide for continuous, sustained release of IL-2. The IL-2/ReGel formulations can also contain various additives, such as polyols including sugars, surfactants, amino acids, other proteins and buffer salts. These additives can serve as functional and/or physical stabilizers of a particular lymphokine, including IL-2, IL-4, IL-12 and their derivatives and mimetics, before injection in the liquid state or at the local IL-2 depot site in the gel after injection.

The formulations of the present invention can be administered once but are preferably administered repeatedly on a daily to monthly basis in order to provide for improved therapeutic results, such as regression in tumor size, thus managing cancer disease chronically or causing complete disappearance or non-reappearance of the tumor. Repeated administration at the same injection site is made possible by the fast removal of the formulation from the injection site over a period of several days to four weeks, depending on the drug carrier type, due to spontaneous, in vivo, chemical degradation of the biodegradable block copolymers.

Generally, the formulation of the present invention can be administered peritumorally or intratumorally at a tumor site that has been identified by a well-established diagnostic imaging technique. One specific route of administration of an IL-2 formulation of the present invention for malignant tumor treatment requiring precise tumor localization is intracranial administration for tumors within the skull. Another specific local route of administration of an IL-2 formulation of the present invention for malignant tumor treatment that does not require specific tumor localization is intraperitoneal IL-2 administration for primary tumors and tumor metastases localized in the peritoneum. Another specific administration route that does not require any sophisticated imaging technique is subcutaneous injection of an IL-2 formulation of the present invention next to tumors localized in the skin, for example primary and metastatic melanoma tumors. Another specific administration route is administration of an IL-2 formulation locally at the site of surgical intervention/tumor excision to kill cancer cells remaining after surgery.

The optimum dose administered will depend on the type of cancer, type of host, tumor localization, route, schedule and sequence of administration, already existing tumor burden (disease state), the type of cytokine, particularly IL-2, IL-4, IL-12 and their derivatives and mimetics, or other lymphokine used. However, the general range is within the range of 1000 I.U. to 1×10⁸ I.U./mL of the drug delivery liquid, i.e., ReGel, for the IL-2-formulations of the present invention.

The method of this invention involves administering to a warm-blooded mammalian host, including a mouse, rat, rabbit, primate, pig or human host, preferably a human patient, a pharmacologically effective amount of a drug, particularly IL-2. This system will not cause unacceptable toxicity or mechanical damage to the surrounding tissue due to the biocompatibility of the materials and pliability of the gel, and will be completely biodegraded to lactic acid, glycolic acid, or other corresponding monomers, and polyethylene glycol within a specific time interval. The IL-2 release rate, gel strength, gelation temperature and degradation rate can be controlled by proper design and preparation of the various drug carriers. For example, the weight percent of A-blocks and B-blocks, the mole percentages of monomers comprising the A-blocks, and the molecular weight and polydispersity of the ABA or BAB triblock copolymers can be modified. IL-2 release is also controllable through adjustment of the concentration and chemical structure of the polymer in the drug delivery liquid.

A dosage form comprised of a polymer solution that contains IL-2, i.e. drug delivery liquid, is administered to the body. This formulation then spontaneously gels, due to the reverse thermal gelation properties of the block copolymer, to form a drug depot as the temperature of the formulation rises to body temperature. In most instances, the IL-2 will make up between about 1000 I.U. to 1×10⁸ I.U./ml of the drug delivery liquid. In some instances, the functionality or physical stability of the interleukins can be increased by addition of various additives to the aqueous solutions or suspensions. Additives, such as polyols (including sugars), amino acids, surfactants, preservatives, antioxidants, stabilizing agents, tonicity adjusting agents, other proteins and certain salts may be used. These additives can be readily incorporated into the drug delivery liquid.

The dosage amount that appears to be most effective is one that results in regression in size of the tumor, complete disappearance or non-reappearance of the tumor, and is not toxic or has an acceptable toxicity to the host. This optimum dose level will depend on many factors, for example, on the type of host and type of cancer, route, schedule and sequence of administration, existing tumor burden, tumor site, the type of IL-2 and the definition of toxicity.

In one embodiment of the present invention, IL-2 (2.0×10⁶ I.U.) is combined with ReGel to form a liquid formulation, which is then injected peritumorally. The IL-2/ReGel forms a gel at the injection site when it is warmed to body temperature and provides for the prolonged release from the depot wherein the IL-2 is released slowly and continuously for several days. The IL-2 that is released from the gel is active and stimulates CTTL's both locally and systemically. In contrast, a single injection of IL-2 in a conventional formulation (i.e., without the ReGel carrier) has been proven ineffective in controlling tumor growth. Therefore, the IL-2/ReGel formulation of the present invention provides local or both local and systemic anticancer therapy from the local injection of a dose of IL-2 that would be sub-therapeutic if administered systemically.

The following examples are presented to illustrate the process of preparing the composition and method of using the composition of the present invention.

### EXAMPLE 1

This example illustrates the in vitro release of IL-2 from the IL-2 formulation of the present invention.

The biodegradable block copolymer carrier in this example is, ReGel, a 23 wt % aqueous solution of a block copolymer having a PLG/PEG-1000 weight ratio of 2.4, a L/G mole ratio of 75/25, a molecular weight of 4,000 Daltons and gelation temperature of 14° C.

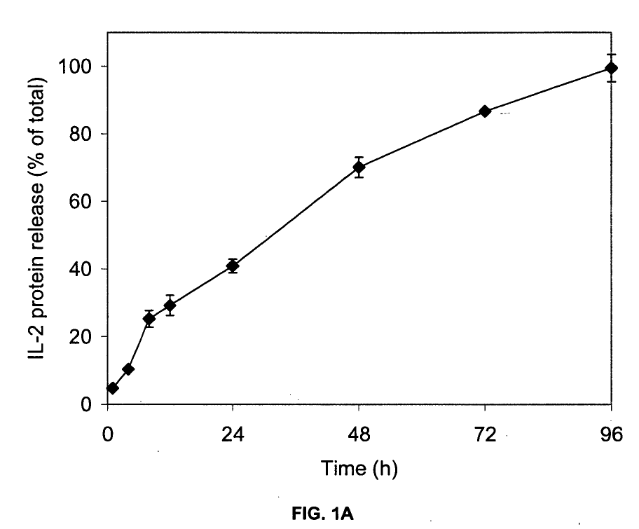

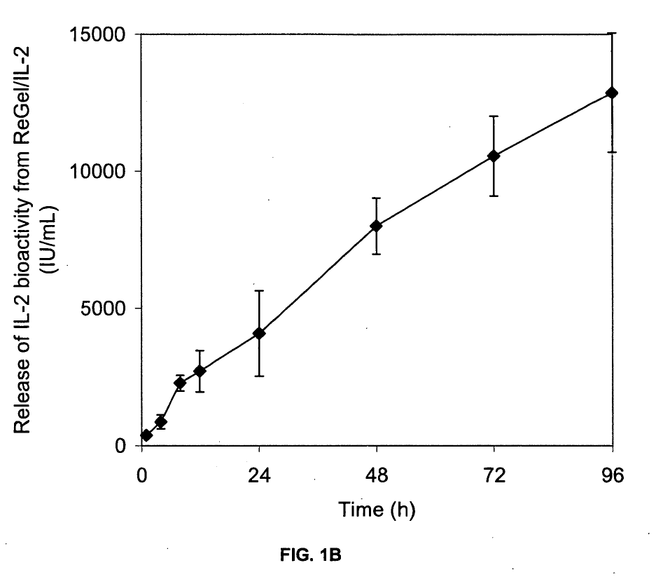

A fixed volume (1 mL) of sterile IL-2 formulation containing 50,000 I.U. (3 μg) of IL-2 (Proleukin®, commercially available from Chiron) was placed into the bottom of 50 mL tissue culture flasks, in duplicate. The flasks were incubated at 37° C. in the presence of 25 mL of tissue culture medium (RPMI 1640, BioWhittaker, Inc.). At pre-selected time points, aliquots (0.5 mL) of release medium were withdrawn and assayed for IL-2 content after appropriate dilution. The IL-2 content was assayed by a specific enzyme linked immunoassay (OptEIA Human IL-2 Set, Pharmingen) and by a quantitative cell proliferative assay using CTTL-20 indicator cells following ³H-thymidine incorporation into the cells (24 hrs incubation at 37° C.). For the latter, a dose response curve was prepared with serial dilutions of standard IL-2. The results were plotted as cumulative IL-2 release as a function of time (FIG. 1A for IL-2 measured by ELISA, FIG. 1B for IL-2 measured by a cell proliferative assay). Both methods showed that IL-2 release was quantitative, lasted 3 to 4 days, and the released IL-2 was fully bioactive.

### EXAMPLE 2

This example illustrates the ability of IL-2 released from the formulations of the present invention to induce cytotoxic lymphocytes by using the same IL-2 formulation as described in Example 1.

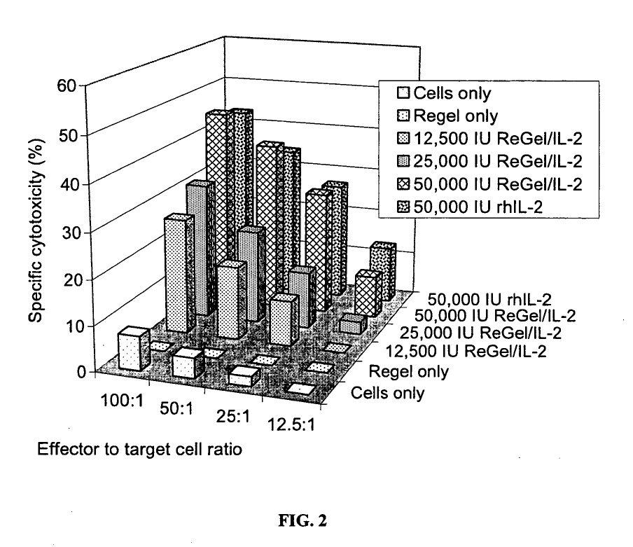

A fixed volume (0.5 mL) of sterile IL-2 formulation as described in Example 1, containing escalating doses of IL-2 (Proleukin®; 12,500 I.U.; 25,000 I.U.; 50,000 I.U.) was placed into the bottom of 25 mL tissue culture flasks, in duplicate. The flasks were incubated at 37° C. for 3 days in the presence of 25 mL of tissue culture medium (RPMI 1640, BioWhittaker, Inc.) containing murine splenocytes. Activated lymphocytes were harvested and analyzed for their capacity to kill RD-995 fibrosarcoma tumor cells in a ⁵¹Cr release assay by determining the percentage (%) of tumor cells undergoing lysis. As shown in FIG. 2, the IL-2 released from ReGel is fully bioactive as compared to free IL-2 added to the release medium at the beginning of the release period. The E:T cell ratio in FIG. 2 represents the ratio of effector cells (activated lymphocytes) to target cells (RD-995 tumor cells), the number of which per sample (10⁴) was kept constant. In conclusion, the IL-2 released from the formulation of the present invention was fully bioactive and the formulation can be used as an effective delivery system for sustained local peritumoral IL-2 delivery in vivo.

### EXAMPLE 3

The example illustrates tumor regression by a single peritumoral injection of the IL-2 formulation of the present invention in mice.

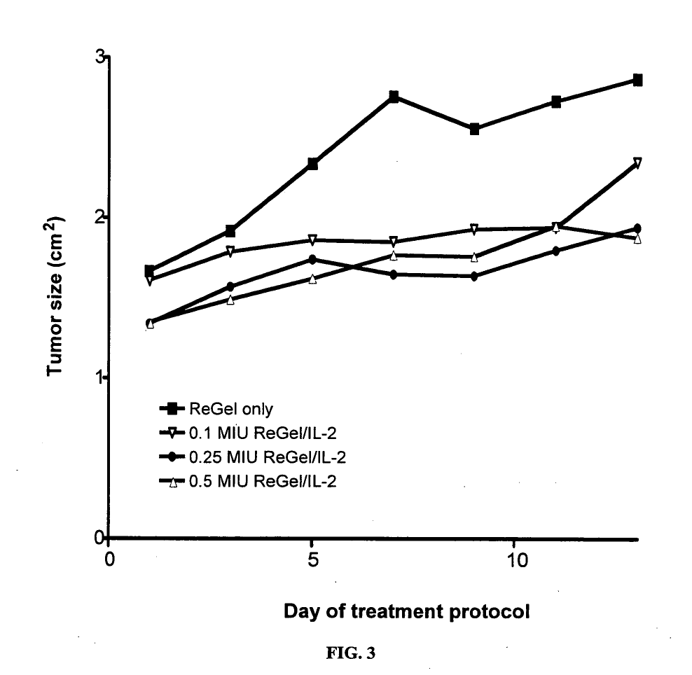

Mice (C3H/HEN) were implanted subcutaneously with RD-995 fibrosarcoma tumor cells. When the solid tumors were 4-5 mm in size, the mice (divided into groups of 6) were injected with 0.2 mL of the IL-2 formulations as described in Example 1, 2×0.1 mL on opposite sides of the tumor perimeter. The IL-2 formulation contained escalating doses of IL-2 100,000 I.U.; 250,000 I.U. or 500,000 I.U. In the control group, the drug carrier alone was injected. Tumor size measurements were obtained every other day for 3 weeks (FIG. 3). Tumor growth was arrested in each group as compared to the control group.

### EXAMPLE 4

This example illustrates the in vitro release of IL-2 from a drug carrier that has a gelation temperature above 20° C.

The biodegradable polymer carrier in this example is a 39 wt % aqueous solution of a block copolymer having a PEG(1000)/PEG(1450) weight ratio of 20/80, a PLG/PEG weight ratio of 2.06, an L/G mole ratio of 85/15, a molecular weight of 4,800 Daltons and gelation temperature of 26° C., and a PEG derivative or a mixture of PEG and a PEG derivative, said PEG or PEG derivative having a weight averaged molecular weight of 150 to 1100 Dalton.

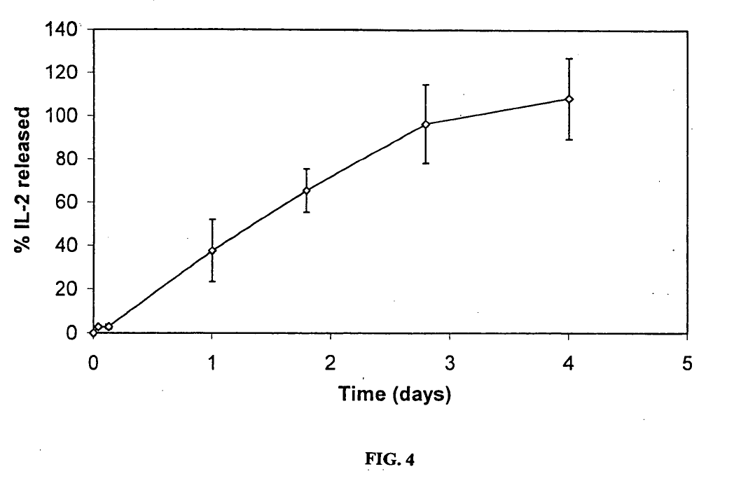

A fixed volume (1 mL) of sterile IL-2 formulation containing 50,000 I.U. (3 μg) of IL-2 (Proleukin®) was placed into the bottom of 50 mL tissue culture flasks, in duplicate. The flasks were incubated at 37° C. in the presence of 25 mL of tissue culture medium (RPMI 1640, BioWhittaker, Inc.). At pre-selected time points, aliquots of release medium (0.5 ml) were withdrawn and after appropriate dilution assayed for IL-2 content by a specific enzyme linked immunoassay (OptEIA Human IL-2 Set, Pharmingen). The results were plotted as IL-2 cumulative release vs. time (FIG. 4). IL-2 loaded in ReGel was released in vitro from the gel depot quantitatively in a bioactive form over a period of 4 days.

### EXAMPLE 5

This example illustrates tumor regression by a single peritumoral injection of an IL-2 formulation in mice. The IL-2 formulation was the same as described in Example 4.

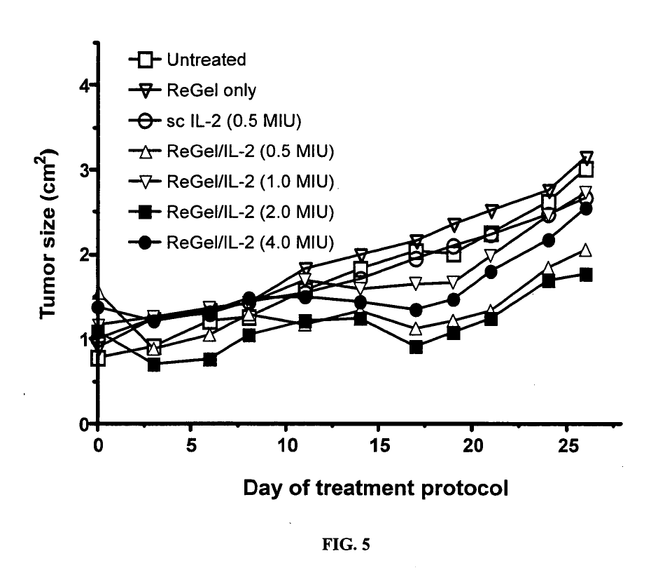

Mice (C3H/HEN) were implanted subcutaneously with RD-995 fibrosarcoma tumor cells. When the solid tumors were 4-5 mm in size, the mice (divided into groups of 10) were injected with 0.2 mL of an IL-2 formulation (2×0.1 mL on opposite sides of the tumor perimeter) containing escalating doses of IL-2 (500,000 I.U.; 1,000,000 I.U.; 2,000,000 I.U. or 4,000,000 I.U.). In the control groups, ReGel only formulations (drug free) or conventional IL-2 formulations (500,000 I.U.) were injected peritumorally. Tumor size measurements were obtained every other day for 26 days (FIG. 5). Tumor growth was arrested for about 3 weeks in each ReGel/IL-2 group as compared to the control groups. Blood pressure was measured on day 3 and 5 after administration in each group of animals. All blood pressure values stayed within the physiological range, and no adverse decreases in blood pressure were detected, indicating that the IL-2 formulation of the present invention caused no adverse side effects at concentrations 4-8 fold greater than the maximum tolerated dose of conventional IL-2 in this animal model.

### EXAMPLE 6

This example illustrates tumor regression by weekly peritumoral injection of an IL-2 formulation in mice. The IL-2 formulation was the same as described in Example 4.

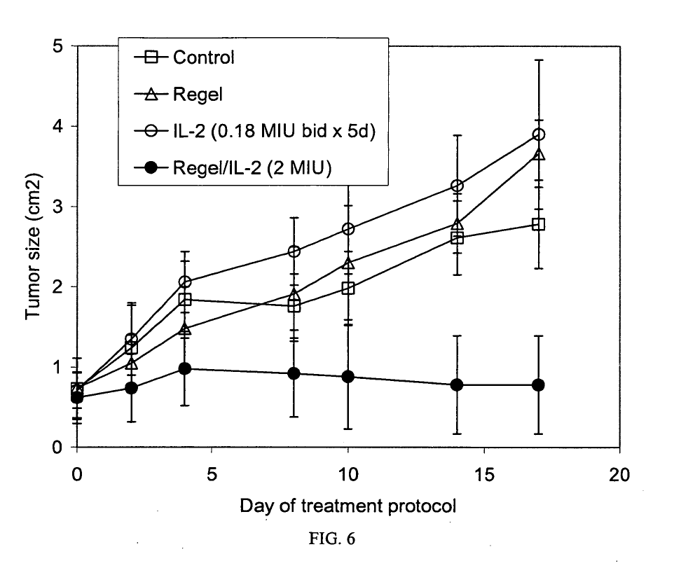

Mice (C3H/HEN) were implanted subcutaneously with RENCA tumor cells. When the solid tumors were about 2×2 mm in size, the mice (divided into groups of 10) were injected with 0.2 mL of an IL-2 formulation (2×0.1 mL on opposite sides of the tumor perimeter) containing 2,000,000 I.U. of IL-2. In the control groups, a drug carrier only formulation (drug free) or a conventional IL-2 formulation (180,000 I.U.) administered systemically (S.C.) twice a day (B.I.D.) for 5 consecutive days (maximum tolerated dose level for systemic IL-2 in mice). The administration was repeated at days 7, 14, and 21 except for the conventional systemic B.I.D. group. Tumor size measurements were obtained every other day (FIG. 6). Tumor growth was arrested for about 2½ weeks in the IL-2 formulation group as compared to the control groups. Blood pressure was measured on day 8 after the first administration in each group of animals. All blood pressure values stayed within the physiological range and no adverse decreases in blood pressure were detected.

### EXAMPLE 7

This example illustrates weekly intraperitoneal injection of an IL-2 formulation in a MethA intraperitoneal tumor mouse model. The IL-2 formulation was the same as described in Example 4.

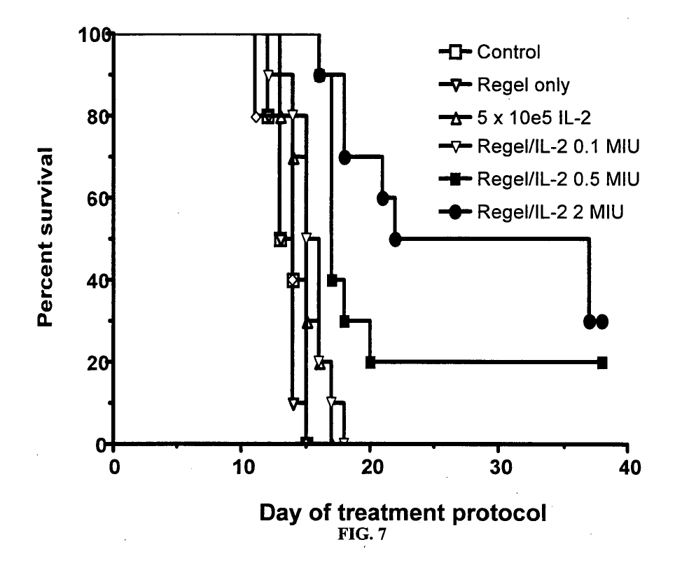

Mice (Balb C) were injected intraperitoneally with 106 MethA fibrosarcoma tumor cells. After 3 days (day 0, FIG. 7), the mice (divided into groups of 10) were injected intraperitoneally with 0.2 mL of an IL-2 formulation containing escalating doses of IL-2 (100,000 I.U.; 500,000 I.U. or 2,000,000 I.U.). The control groups consisted of tumor bearing untreated mice, drug free formulation and mice injected intraperitoneally with 500,000 I.U. of conventional IL-2. The injections were repeated weekly at days 7, 14, 21, 28 and 35. Mice were followed for survival (FIG. 7). It was demonstrated that the IL-2 formulation of the present invention gave superior survival data as compared to the positive controls using conventional IL-2 formulations.

### EXAMPLE 8

This example illustrates survival rate by weekly peritumoral injection of the IL-2 formulation of the present invention in mice bearing B16 melanoma solid tumor. The IL-2 formulation was the same as described in Example 4.

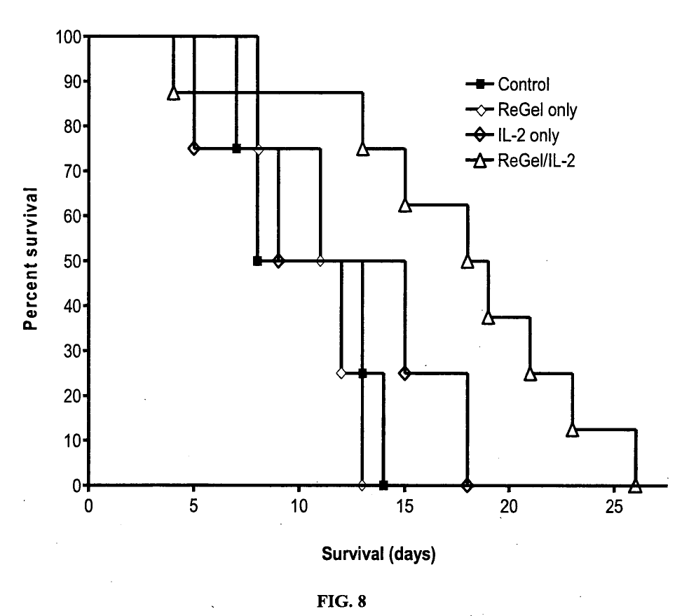

Mice (C57/B16) were implanted subcutaneously with B16 melanoma tumor cells. When the solid tumors were 4-5 mm in size, the mice (divided into groups of 8) were injected with 0.2 mL of the IL-2 formulation of the present invention (2×0.1 mL on opposite sides of the tumor perimeter) containing 2,000,000 I.U. of IL-2. In the control groups, drug carrier only formulations (drug free) or conventional IL-2 formulations (2,000,000 I.U.) were injected peritumorally. The administration was repeated at day 7, 14, and 21, 28 and 35. Mice were followed for tumor growth and survival rate. Blood pressure was measured on day 1, 2, 3, 4 and 5 (after the first administration), and repeated on day 14, 15, 16, 17 and 18 (after the third administration) in each animal group. All blood pressure values stayed within the physiological range and no adverse decreases in blood pressure were detected. While tumor growth was significantly slowed with the IL-2 formulation of the present invention as compared to the control groups, the IL-2 formulation of the present invention provided superior survival data as compared to the positive control group using a conventional IL-2 formulation (FIG. 8).

### EXAMPLE 9

This example illustrates activation of systemic immunity by weekly peritumoral injection of an IL-2 formulation in mice, the drug carrier is the same as described in Example 1.

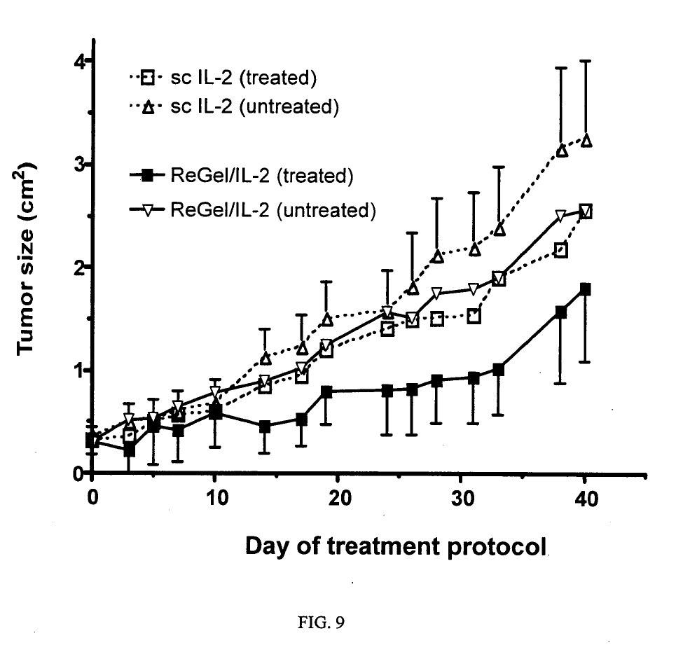

Mice (C3H/HEN) are implanted subcutaneously with two tumors, one tumor on each flank using RD-995 fibrosarcoma tumor cells. When the solid tumors are 4-5 mm in size, the mice (divided into groups of 10) are injected with a 0.2 mL formulation as described in Example 1, 2×0.1 mL on opposite sides of the tumor perimeter containing 2,000,000 I.U. of IL-2 on only right sided tumor (5 mice) or left sided tumor (5 mice) bearing mice. The control groups consisted of untreated mice bearing two tumors and mice injected peritumorally (5 mice with left sided tumors and 5 mice with right sided tumors) with conventional IL-2 formulations (2,000,000 I.U.). In each group, administrations are repeated weekly at identical pre-selected tumor sites for 8 consecutive weeks. Growth of both treated as well as untreated tumors is arrested (or the tumors regressed completely) in the IL-2/formulation group while in the control groups, both tumors grow progressively. Accordingly, survival data for the IL-2 formulation group are superior to those obtained for the control groups, including the peritumoral conventional IL-2 group, demonstrating that a systemic immune response is only achieved with the IL-2 formulation of the present invention (FIG. 9).

### EXAMPLE 10

This example illustrates the ability of ReGel to alter the pharmacokinetic profile of IL-2 by using the same IL-2 formulation as described in Example 4.

A fixed volume (0.1 mL) of sterile IL-2 formulation as described in Example 4, containing 2×10⁶ IU ReGel/IL-2 containing approximately 290,000 CPM ¹²⁵I-labelled IL-2 (split into 4 quadrant injections into subcutaneous tissue adjacent to mice implanted with RD-995 tumors) was treated with peritumoral injections in mice previously implanted with 1×10⁶ RD-995 cells. The mice were anesthetized and blood samples were obtained from the axillary artery from cohorts of 3 mice at 0.17, 0.5, 1, 2, 4, 8, 12, 24, 48, 72, 96 and 120 h, after which the mice were sacrificed. Samples (100 μl blood) from each mouse were analyzed for IL-2 by Gamma counter (R&D Scientific). The tumor site and kidneys of the mice were also excised and analyzed for retention of radiolabeled [¹²⁵I] IL-2. In another experiment, groups of 4 mice were acclimated to metabolic cages and treated with either ReGel/IL-2 (as described above) or peritumoral injection of recombinant human IL-2 (a dose of 2,000,000 IU IL-2 containing ˜290,000 CPM ¹²⁵I-labeled IL-2 via a 4-quadrant technique). Sequential 24-hour collections of urine and stool were performed on these mice (pooled from subsets of 4 mice in metabolic cages).

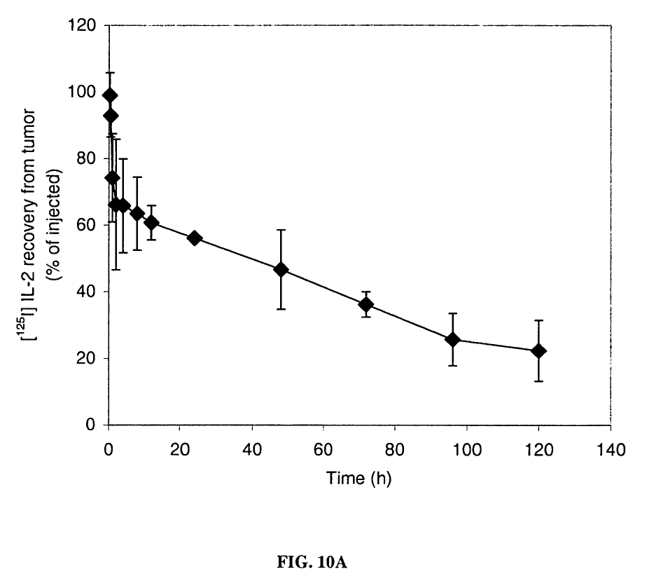

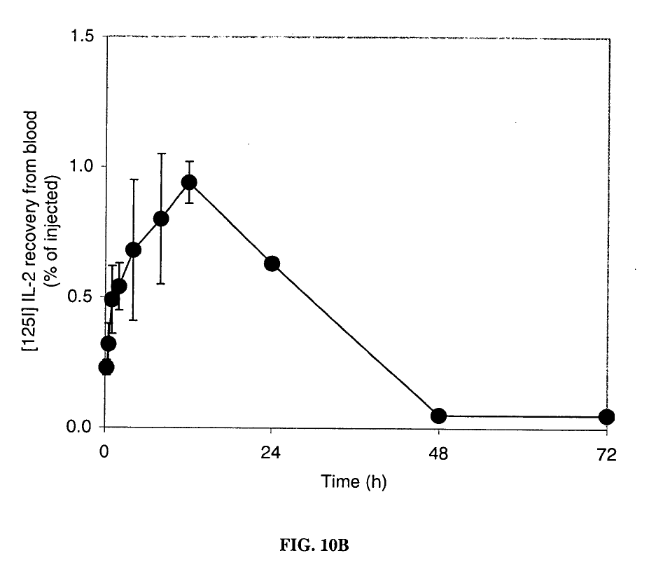

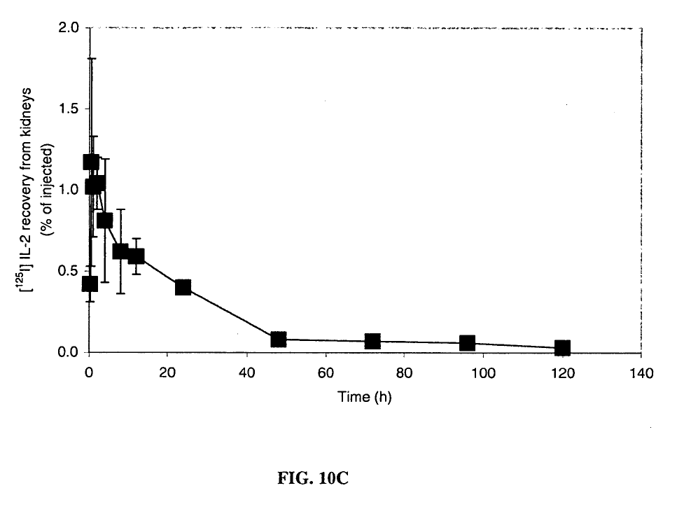

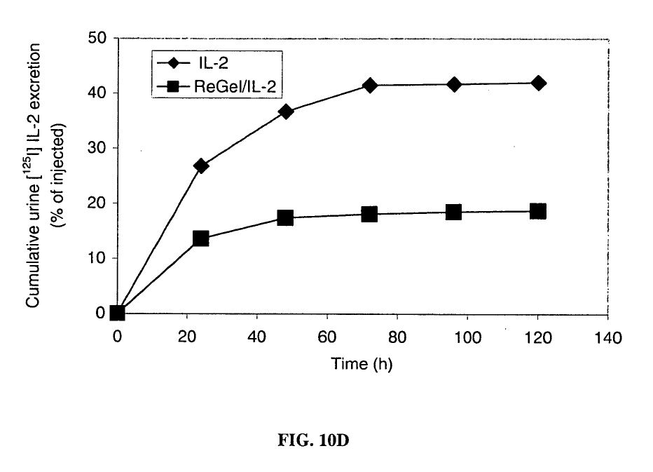

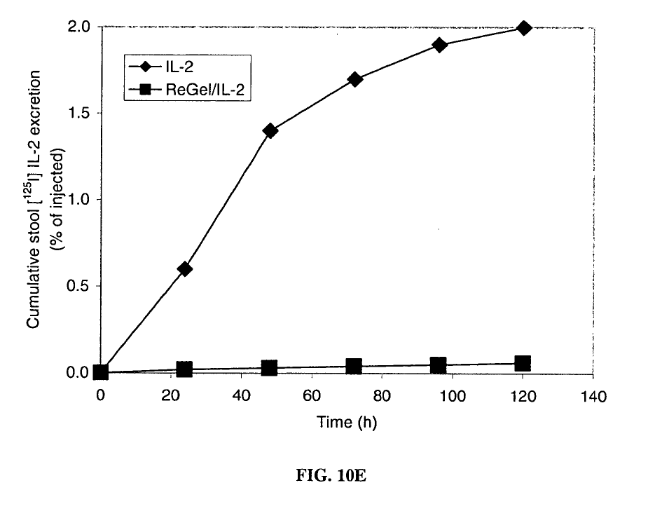

This experiment demonstrated an approximately 96% recovery of injected radioactivity after 15 minutes at the tumor site. Retention of IL-2 bound radioactivity at the tumor site appeared to decline in a biphasic fashion (FIG. 10A). There was a rapid initial burst release (approximately 25% of the injected dose) in the first hour, followed by a more gradual nearly linear decline between 1 and 96 h (T_{1/2}β approximately 48 h). It appears that about 25% of the injected IL-2 remained detectable at the tumor site for over 96 h. Very little IL-2 (Cₘₐₓ<1.5% of injected dose) was recovered from blood (FIG. 10B) or kidney (FIG. 10C) of the experimental mice within the first 48 h. The blood levels peaked at 12 h, while renal tissue levels peaked in the first hour and declined thereafter. After 48 h, no IL-2 was detectable in the blood or kidney.

Sequential 24-hour collections of urine and stool were performed on groups of 4 mice in metabolic cages. Cumulative urine excretion of IL-2 from ReGel reached approximately 18% of the total injected radioactivity by 72 h (FIG. 10D). In contrast, approximately 42% of free IL-2-derived radioactivity was excreted in the urine over the same time. There was negligible excretion of IL-2-derived radioactivity in the stool of animals receiving ReGel/IL-2 (<0.1%), while mice receiving recombinant IL-2 had approximately 2% of the dose recovered from their stool within 120 h (FIG. 10E). The results of these experiments demonstrate prolonged retention of IL-2 at the tumor site, with a Cₘₐₓ less than 1.5% of the dose entering the circulation. The low cumulative recovery of IL-2 from urine and stool from ReGel/IL-2 injected mice suggests that IL-2 is retained and metabolized at the site of injection. This is in contrast to subcutaneous or soft tissue injections of IL-2, which have a half life of 5.1 to 5.2 h (Eton O, Rosenblum M G, Legha S S, et al. Phase I trial of subcutaneous recombinant human interleukin-2 in patients with metatstatic melanoma. Cancer 2002;95(1):127-34, and Kirchner GI, Franzke A, BuerJ, et al. Pharmacokinetics of recombinant human interleukin-2 in advanced renal cell carcinoma patients following subcutaneous application. Br J Clin Pharmacol 1998;46(1):5-10).

### EXAMPLE 11

This example illustrates the ability of ReGel to alter the T cell recruitment profile of IL-2 by using the same IL-2 formulation as described in Example 4.

Mice (C3H/HEN) were implanted subcutaneously with RENCA tumor cells. When the solid tumors were about 2×2 mm in size, the mice (divided into groups of 10) were injected with 0.2 mL of an IL-2 formulation (2×0.1 mL on opposite sides of the tumor perimeter) containing 2,000,000 I.U. of IL-2. In the control groups, a drug carrier only formulation (drug free). The administration was repeated at days 7, 14, and 21. Two mice from each tumor group were sacrificed after the 4^{th} weekly injection. Tumors were embedded in paraffin and used to prepare frozen sections to be immunostained with CD3 (pan T cell marker), CD8 (cytotoxic T cells), and NK-1 (NK cells) to determine changes in the immune response within the tumor. The slides were scored on a 1-4+ scale (Table1). This staining demonstrated increased CD8+/CD3+ T-cell and NK-1 (NK cells) infiltration into tumors treated with ReGel/IL-2 versus untreated control and conventional IL-2.

TABLE 1
Immunohistologic staining of RENCA tumors
following ReGel/IL-2 treatment

| | CD-3 | CD-8 | NK |
|---|---|---|---|
| Control | 1+ | 2+ | neg |
| IL-2 Only | 2+ | 3+ | neg |
| ReGel Only | 1+ | 2+ | ± |
| ReGel + IL-2 | 3+ | 3+ | 1+ |

### EXAMPLE 12

This example illustrates survival rate by a single intracranial, peritumoral injection of the IL-2 formulation of the present invention in mice bearing B16 melanoma solid tumor. The IL-2 formulation was the same as described in Example 4.

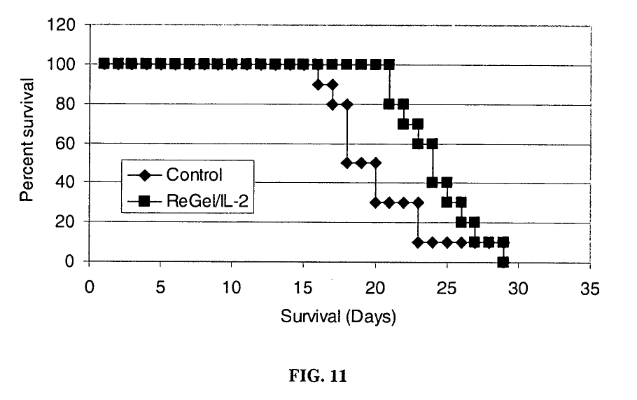

This example also illustrates the ability of ReGel to deliver IL-2 in a controlled release formulation in areas where a bolus release of IL-2 would result in undesirable side effects, i.e., brain. Mice were inoculated intracranially with the B16-F10 melanoma cell line and subsequently 5 μL of IL-2/ReGel at a concentration of up to 9.2×10⁶ IU IL-2/mL were injected into the tumor site. Mice were evaluated for inflammation/neurotoxicity, T-cell infiltration, and survival. Inflammation following ReGel/IL-2 injection was reduced in comparison to administration of conventional IL-2 and there was no evidence of neurotoxicity. T-cell infiltration was dose dependent, with 5 μL doses of 4.6 and 9.2×10⁶ IU of IL-2/mL/ReGel inducing pronounced infiltration. Survival was statistically enhanced, versus the untreated control. Median survival was 24 days versus 17 days, for ReGel/IL-2 versus untreated control, respectively. FIG. 11

### EXAMPLE 13

The biodegradable block copolymer carriers in this example is a 25 wt % aqueous solution of BAB triblock ReGel copolymer having a PLG/PEG weight ratio of 2.6, a L/G mole ratio of 78/22, a molecular weight of 4,900 Daltons and a gelation temperature of 25° C., or a combination of ReGel, from example 1, and BAB ReGel from this example at various ratios.

This example illustrates a configuration to reduced initial release and/or prolong the overall duration of cytokine release.

A fixed volume (1 mL) of sterile cytokine formulation containing 50,000 I.U. (3 μg) of cytokine is placed into the bottom of 50 mL tissue culture flasks, in duplicate. The flasks are incubated at 37° C. in the presence of 25 mL of tissue culture medium (RPMI 1640, BioWhittaker, Inc.). At pre-selected time points, aliquots (0.5 mL) of release medium are withdrawn and assayed for cytokine content after appropriate dilution. The cytokine content is assayed by a specific enzyme linked immunoassay and by a quantitative cell proliferative assay using indicator cells following ³H-thymidine incorporation into the cells (24 hrs incubation at 37° C.). For the latter, a dose response curve is prepared with serial dilutions of standard cytokine. The results are plotted as cumulative cytokine release as a function of time. Both methods showed that cytokine release is quantitative and the released cytokine is fully bioactive.

The varied configurations of the present invention using BAB ReGel or combinations of ReGel and BAB ReGel generated cytokine release profiles with initial levels <10% in 1 h to 2 days and/or prolonged the duration of release such that greater than 70% of the cytokine is released between day 2 and day 90.

The above Examples are presented for illustrative purposes only, and are not intended, and should not be construed to limit the invention in any manner. Various modifications of the compounds and methods of the invention may be made without departing from the spirit or scope thereof. It will be immediately apparent to one skilled in the art which various modifications may be made without departing from the scope of the invention that is limited only by the following claims and their functional equivalents.

1. An aqueous formulation for local administration of an IL-2 to a warm blooded animal to provide a local or both a local and systemic therapeutic effect, comprising:
a) 1000 I.U. to 1×10⁸ I.U./ml of an IL-2, and
b) 5 to 50 wt % of a biodegradable polymeric drug carrier,
wherein said formulation can be administered intratumorally/peritumorally and form an IL-2 containing depot with a pharmacokinetic(PK) profile defined by:
i) an initial release of IL-2 from the depot of between 0.1 and 50% of the administered dose,
ii) an IL-2 release duration from the formulation within the range from 1-90 days, and
iii) an intratumoral half-life of IL-2 of from 6 hours to 6 weeks.

2. The formulation according to claim 1, wherein the initial release of IL-2 from the depot is between 5-40%, the IL-2 release duration from the system is within the range 3-60 days, and the plasma half-life of IL-2 is 12 hours to 4 weeks.

3. The formulation according to claim 2, wherein the initial release of IL-2 from the depot is between 10-30%, the IL-2 release duration from the system is within the range from 5-30 days and the plasma half-life of IL-2 is from 18 hours to 2 weeks.

4. The formulation according to claim 1, wherein the PK is further defined by a urine excretion of IL-2 after administration which is below 30%.

5. The formulation according to claim 1, wherein the PK is further defined by an excretion of IL-2 via the fecal route of less than 0.5%.

6. The formulation according to claim 1, wherein said biodegradable polymeric drug carrier comprises a biodegradable ABA- or BAB-type tri-block copolymer comprising:
i) 51 to 83% by weight of a biodegradable, hydrophobic A block comprising a biodegradable polyester or poly(ortho ester), and
ii) 17 to 49% by weight of a hydrophilic B block comprising a polyethylene glycol (PEG),
said tri-block copolymer having a weight average molecular weight of between 2000 to 4990 Daltons and possessing reverse thermal gelation properties.

7. The formulation according to claim 1, wherein the IL-2 are interleukin-2 (IL-2) derivatives or IL-2 mimetics.

8. The formulation according to claim 1, wherein the formulation is an injectable liquid prior to administration.

9. The formulation according to claim 1, further comprising a biocompatible additive selected from the group consisting of polyols including sugars, surfactants, amino acids, proteins, preservatives, antioxidants, stabilizing agents, tonicity adjusting agents, buffer salts and equivalents thereof.

10. The formulation according to claim 1, further comprising a reconstitution enhancing and enabling agent comprising a polyethylene glycol (PEG), a PEG derivative or a mixture of PEG and a PEG derivative, said PEG or PEG derivative having a weight averaged molecular weight of 150 to 1100 Daltons.

11. The formulation according to claim 10, wherein the PEG derivative is comprised of PEG and a member selected from the group consisting of D,L-lactide, D-lactide, L-lactide, D,L-lactic acid, D-lactic acid, L-lactic acid, glycolide, glycolic acid and copolymers thereof.

12. The formulation according to claim 10, wherein the PEG derivative is represented by R¹—CO—O—(PEG)-CO—R² or R¹—O-(PEG)-R² and wherein R¹ and R² are independently members selected from the group consisting of H and C₁ to C₁₀ alkyl.

13. An aqueous formulation for local administration of a lymphokine to a warm blooded animal to provide local or both local and systemic therapeutic effects, comprising:
An effective amount of a lymphokine and 5 to 50 wt % of a biodegradable polymeric drug carrier, wherein said formulation can be administered intratumorally/peritumorally and form a lymphokine containing depot with a pharmacokinetic (PK) profile defined by:
i) an initial release of lymphokine from the depot of between 0.1 and 50% of the administered dose,
ii) an lymphokine release duration from the formulation within the range from 1-90 days, and
iii) a plasma half-life of lymphokine of from 6 hours to 6 weeks.

14. The formulation according to claim 13, wherein the lymphokine is a member selected from the group consisting of interleukin-2 (IL-2), interleukin-4, interleukin-12, derivatives and mimetics thereof.

15. The formulation according to claim 13, wherein the initial release of lymphokine from the depot is between 5-40%, the lymphokine release duration from the system is within the range 3-60 days, and the plasma half-life of lymphokine is 12 hours to 4 weeks.

16. The formulation according to claim 15, wherein the initial release of lymphokine from the depot is between 10-30%, the lymphokine release duration from the system is within the range from 5-30 days and the plasma half-life of lymphokine is from 18 hours to 2 weeks.

17. The formulation according to claim 13, wherein the PK is further defined by a urine excretion of lymphokine after administration which is below 30%.

18. The formulation according to claim 13, wherein the PK is further defined by an excretion of lymphokine via the fecal route of less than 0.5%.

19. The formulation according to claim 13, wherein said biodegradable polymeric drug carrier comprising a biodegradable ABA- or BAB-type tri-block copolymers comprising:
i) 51 to 83% by weight of a biodegradable, hydrophobic A block comprising a biodegradable polyester or poly(ortho ester), and
ii) 17 to 49% by weight of a hydrophilic B block comprising a polyethylene glycol (PEG), said tri-block copolymer having a weight average molecular weight of between 2000 to 4990 Daltons and possessing reverse thermal gelation properties.

20. The formulation according to claim 13, further comprising a reconstitution enhancing and enabling agent comprising a polyethylene glycol (PEG), a PEG derivative or a mixture of PEG and a PEG derivative, said PEG or PEG derivative having a weight averaged molecular weight of 150 to 1100 Daltons.

21. The formulation according to claim 13, where the formulation is an injectable liquid prior to administration.

22. The formulation according to claim 13, further comprising a biocompatible additive selected from the group consisting of polyols including sugars, surfactants, amino acids, proteins, preservatives, antioxidants, stabilizing agents, tonicity adjusting agents, buffer salts and equivalents thereof.

23. The formulation according to claim 20, wherein the PEG derivative is comprised of PEG and a member selected from the group consisting of D,L-lactide, D-lactide, L-lactide, D,L-lactic acid, D-lactic acid, L-lactic acid, glycolide, glycolic acid and copolymers thereof.

24. The formulation according to claim 20, wherein the PEG derivative is represented by R¹—CO—O-(PEG)-CO—R¹ or R¹—O-(PEG)-R² and wherein R¹ and R² are independently members selected from the group consisting of H and C₁ to C₁₀ alkyl groups.

25. A method for the local or both local and systemic control of proliferative cell disorders in a warm-blooded animal, comprising:
a) preparing an aqueous lymphokine formulation comprising an effective amount of a lymphokine; and one or more biodegradable ABA- or BAB-type tri-block copolymers comprising:
i) 51 to 83% by weight of a biodegradable, hydrophobic A block comprising a biodegradable polyester or poly(ortho ester), and
ii) 17 to 49% by weight of a hydrophilic B block comprising a polyethylene glycol (PEG), said tri-block copolymer having a weight average molecular weight of between 2000 to 4990 Daltons and possessing reverse thermal gelation properties;
b) administering said formulation adjacent or into the area of said warm-blooded animal where the proliferate cell disorder occurs;
c) allowing said formulation to form a lymphokine containing depot which provides for continuous, sustained release of the lymphokine such that local or both local and systemic therapeutic effects are achieved without causing unacceptable side effects.

26. The method according to claim 25, wherein the lymphokine containing depot has a pharmacokinetic (PK) profile defined by:
i) an initial release of lymphokine from the depot of between 0.1 and 50% of the administered dose,
ii) a lymphokine release duration from the formulation within the range from 1-90 days, and
iii) a plasma half-life of lymphokine of from 6 hours to 6 weeks.

27. The method according to claim 26, wherein the PK is further defined by a urine excretion of lymphokine after administration which is below 30%.

28. The method according to claim 26, wherein the PK is further defined by an excretion of lymphokine via the fecal route of less than 0.5%.

29. The method according to claim 25, wherein the lymphokine is a member selected from the group consisting of interleukin-2 (IL-2), interleukin-4, interleukin-12, derivatives and mimetics thereof.

30. The method according to claim 25, wherein the formulation is an injectable liquid prior administration.

31. The method according to claim 25, wherein the formulation further comprises a biocompatible additive selected from the group consisting of polyols including sugars, surfactants, amino acids, proteins, preservatives, antioxidants, stabilizing agents, tonicity adjusting agents, buffer salts and equivalents thereof.

32. The method according to claim 25, wherein the proliferative cell disorder is a cancer or warts.

33. The method according to claim 25, wherein the administration is via a parenteral means.

34. The method according to claim 33, wherein the parenteral means is a member selected from the group consisting of intratumoral, peritumoral, perilesional, intralesional, intrathecal, intracranial, intraperitoneal, and intra-abdominal.

35. The method according to claim 25, wherein the formulation is administered daily to every three months.

36. The method according to claim 25, wherein the formulation further comprises a reconstitution enhancing and enabling agent comprising a polyethylene glycol (PEG), a PEG derivative or a mixture of PEG and a PEG derivative, said PEG or PEG derivative having a weight averaged molecular weight of 150 to 1100 Daltons.

37. The method according to claim 36, wherein the PEG derivative is comprised of PEG and a member selected from the group consisting of D,L-lactide, D-lactide, L-lactide, D,L-lactic acid, D-lactic acid, L-lactic acid, glycolide, glycolic acid and copolymers thereof.

38. The method according to claim 27, wherein the PEG derivative is represented by R¹—CO—O-(PEG)-CO—R² or R¹—O-(PEG)-R² and wherein R¹ and R² are independently members selected from the group consisting of H and C₁ to C₁₀ alkyl groups.

39. A method for local or both local and systemic control of proliferative cell disorders, comprising:
a) preparing an IL-2 formulation from an effective amount of an IL-2; and one or more biodegradable ABA- or BAB-type tri-block copolymers comprising:
i) 51 to 83% by weight of a biodegradable, hydrophobic A block comprising a biodegradable polyester or poly(ortho ester), and
ii) 17 to 49% by weight of a hydrophilic B block comprising a polyethylene glycol (PEG), said tri-block copolymer having a weight average molecular weight of between 2000 to 4990 Daltons and possessing reverse thermal gelation properties;
b) administering said formulation adjacent or into the area of a warm blooded animal where the proliferate cell disorder occurs;
c) allowing said formulation to form an IL-2 containing depot which provides continuous, sustained release of IL-2 such that local or both local and systemic therapeutic effects are achieved without causing unacceptable side effects.

40. The method according to claim 30, wherein the IL-2 containing depot has a pharmacokinetic (PK) profile defined by:
i) an initial release of IL-2 from the depot of between 0.1 and 50% of the administered dose,
ii) an IL-2 release duration from the formulation within the range from 1-90 days, and
iii) a plasma half-life of IL-2 of from 6 hours to 6 weeks.

41. The method according to claim 40, wherein the PK is further defined by a urine excretion of IL-2 after administration which is below 30%.

42. The method according to claim 41, wherein the PK is further defined by an excretion of IL-2 via the fecal route of less than 0.5%.

43. The method according to claim 39, wherein the IL-2 is an IL-2 derivative or IL-2 mimetic.

44. The method according to claim 39, wherein the formulation is an injectable liquid prior to administration.

45. The method according to claim 39, wherein the formulation further comprises a biocompatible additive selected from the group consisting of polyols, sugars, surfactants, amino acids, proteins, preservatives, antioxidants, stabilizing agents, tonicity adjusting agents, buffer salts and equivalents thereof.

46. The method according to claim 39, wherein the proliferative cell disorder is a cancer or warts

47. The method according to claim 39, wherein the administration is via a parenteral means.

48. The method according to claim 47, wherein the parenteral means is a member selected from the group consisting of intratumoral, peritumoral, perilesional, intralesional, intrathecal, intracranial, intraperitoneal, and intra-abdominal.

49. The method according to claim 39, wherein the formulation is administered daily to monthly.

50. The method according to claim 39, wherein the IL-2 content in said formulation is within the range of 1000 to 1×10⁸ I.U./ml.

51. The method according to claim 39, wherein the formulation further comprises a reconstitution enhancing and enabling agent comprising a polyethylene glycol (PEG), a PEG derivative or a mixture of PEG and a PEG derivative, said PEG or PEG derivative having a weight averaged molecular weight of 150 to 1100 Daltons.

52. The method according to claim 51, wherein the PEG derivative is comprised of PEG and a member selected from the group consisting of D,L-lactide, D-lactide, L-lactide, D,L-lactic acid, D-lactic acid, L-lactic acid, glycolide, glycolic acid and copolymers thereof.

53. The method according to claim 52, wherein the PEG derivative is represented by R¹—CO—O-(PEG)-CO—R² or R¹—O-(PEG)-R² and wherein R¹ and R² are independently members selected from the group consisting of H and C₁ to C₁₀ alkyl groups.

54. A method for treating proliferative cell disorders within the brain comprising:
a) preparing an aqueous lymphokine formulation comprising an effective amount of a lymphokine; and one or more biodegradable ABA- or BAB-type tri-block copolymers comprising:
i) 51 to 83% by weight of a biodegradable, hydrophobic A block comprising a biodegradable polyester or poly(ortho ester), and
ii) 17 to 49% by weight of a hydrophilic B block comprising a polyethylene glycol (PEG), said tri-block copolymer having a weight average molecular weight of between 2000 to 4990 Daltons and possessing reverse thermal gelation properties;
b) administering intracranially said formulation adjacent or into the area of said warm-blooded animal where the proliferate cell disorder occurs; and
c) allowing said formulation to form a lymphokine containing depot within the brain which provides for continuous, sustained release of the lymphokine such that local or both local and systemic therapeutic effects are achieved without causing unacceptable side effects.

55. The method according to claim 54, wherein the lymphokine containing depot has a pharmacokinetic (PK) profile defined by:
i) an initial release of lymphokine from the depot of between 0.1 and 50% of the administered dose,
ii) an lymphokine release duration from the formulation within the range from 1-90 days, and
iii) a plasma half-life of lymphokine of from 6 hours to 6 weeks.

56. The method according to claim 54, wherein the PK is further defined by a urine excretion of IL-2 after administration which is below 30%.

57. The method according to claim 54, wherein the PK is further defined by an excretion of IL-2 via the fecal route of less than 0.5%.

58. The method according to claim 54, wherein the lymphokine is a member selected from the group consisting of interleukin-2 (IL-2), interleukin-4, interleukin-12, derivatives and mimetics thereof.

59. The method according to claim 54, wherein the formulation is an injectable liquid prior administration.

60. The method according to claim 54, wherein the formulation further comprises a biocompatible additive selected from the group consisting of polyols including sugars, surfactants, amino acids, proteins, preservatives, antioxidants, stabilizing agents, tonicity adjusting agents, buffer salts and equivalents thereof.

61. The method according to claim 54, wherein the proliferative cell disorder is cancer.

62. The method according to claim 54, wherein the formulation is administered every three months.

63. The method according to claim 54, wherein the formulation further comprises a reconstitution enhancing and enabling agent comprising a polyethylene glycol (PEG), a PEG derivative or a mixture of PEG and a PEG derivative, said PEG or PEG derivative having a weight averaged molecular weight of 150 to 1100 Daltons.

64. The method according to claim 63, wherein the PEG derivative is comprised of PEG and a member selected from the group consisting of D,L-lactide, D-lactide, L-lactide, D,L-lactic acid, D-lactic acid, L-lactic acid, glycolide, glycolic acid and copolymers thereof.

65. The method according to claim 63, wherein the PEG derivative is represented by R¹—CO—O-(PEG)-CO—R² or R¹—O-(PEG)-R and wherein R¹ and R² are independently members selected from the group consisting of H and C₁ to C₁₀ alkyl groups.
